(19) United States
(12) Patent Application Publication
(10) Pub. No.: US 2023/0168647 A1
(43) Pub. Date: Jun. 1, 2023

(54) COGNITIVE PERFORMANCE DETERMINATION BASED ON INDOOR AIR QUALITY

(71) Applicant: Airsset Technologies Inc., Vancouver (CA)

(72) Inventors: Zoe Le Hong, Vancouver (CA); Matthew Canavan, Vancouver (CA); Michael Driedger, Vancouver (CA); Marcos Moreno, Vancouver (CA)

(73) Assignee: Airsset Technologies Inc., Vancouver (CA)

(21) Appl. No.: 17/537,148

(22) Filed: Nov. 29, 2021

(51) Int. Cl. G05B 19/042

(52) U.S. Cl. CPC G05B 19/042; G05B 2219/2614

(57) ABSTRACT

Systems and methods of environmental parameter determination are provided. A system can include a data processing system to obtain sensor data, apply a data normalization operation to the sensor data to generate a normalized data set for storage in a database, obtain, from the database, the normalized data set to generate at least a first cognitive index and a second cognitive index, each of the first cognitive index and the second cognitive index, compare each of the first cognitive index and the second cognitive index with a threshold, generate, based on the first cognitive index and the second cognitive index, a unified cognitive index for the indoor space, generate, responsive to the unified cognitive index, a digital output that corresponds to the unified cognitive index; and provide, from the data processing system, the digital output to a client computing device for display by the client computing device.

### BACKGROUND

Environmental parameters can be measured with respect to spaces, such as indoor spaces in buildings. These parameters can affect the performance of occupants present in the spaces.

### SUMMARY

At least one aspect is directed to a system of environmental parameter determination in an indoor environment. The system can include a data processing system including memory and at least one processor to obtain, via a network and from a first sensor, first indoor air composition data that indicates a first metric of an indoor space; obtain, via the network and from a second sensor, second indoor air composition data that indicates a second metric of the indoor space; obtain, via the network and from a third sensor, third indoor air composition data that indicates a third metric of the indoor space; apply a data normalization operation to at least one of the first indoor air composition data, the second indoor air composition data, and the third indoor air composition data to generate a normalized data set for storage in a database, the normalized data set including the first indoor air composition data, the second indoor air composition data, and the third indoor air composition data; obtain, from the database, the normalized data set to generate at least a first cognitive index and a second cognitive index, each of the first cognitive index and the second cognitive index corresponding to one of the first metric of the indoor space, the second metric of the indoor space, or the third metric of the indoor space; compare each of the first cognitive index and the second cognitive index with a threshold; generate, based on the first cognitive index and the second cognitive index, a unified cognitive index for the indoor space; generate, responsive to the unified cognitive index, a digital output that corresponds to the unified cognitive index; and provide, from the data processing system, the digital output to a client computing device for display by the client computing device.

At least one aspect is directed to a method of environmental parameter determination in an indoor environment. The method can include receiving, by a data processing system including memory and at least one processor, from a first sensor, first indoor air composition data that indicates a first metric of an indoor space; receiving, by the data processing system, from second first sensor, second indoor air composition data that indicates a second metric of an indoor space; receiving, by the data processing system, from a third sensor, third indoor air composition data that indicates a first metric of an indoor space; applying a data normalization operation to at least one of the first indoor air composition data, the second indoor air composition data, and the third indoor air composition data to generate a normalized data set for storage in a database, the normalized data set including the first indoor air composition data, the second indoor air composition data, and the third indoor air composition data; generating, based on the normalized data set retrieved, from the database, at least a first cognitive index and a second cognitive index, each of the first cognitive index and the second cognitive index corresponding to one of the first metric of the indoor space, the second metric of the indoor space, or the third metric of the indoor space; evaluating each of the first cognitive index and the second cognitive index against a threshold; generating, based on the first cognitive index and the second cognitive index, a unified cognitive index for the indoor space; generating, responsive to the unified cognitive index, a digital output corresponding to the unified cognitive index; and providing, from the data processing system, the digital output to a client computing device for display by the client computing device.

These and other aspects and implementations are discussed in detail below. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIG. 1 depicts an example system to perform environmental parameter determination, in accordance with an implementation.

FIG. 2 depicts an example digital output of a unified cognitive index and indoor air composition data, in accordance with an implementation.

FIG. 3 depicts an example method of environmental parameter determination, in accordance with an implementation.

FIG. 4 depicts an example computing system, in accordance with an implementation.

### DETAILED DESCRIPTION

Following below are more detailed descriptions of various concepts related to, and implementations of, methods, apparatuses, and systems of environmental parameter determination in an indoor environment. The various concepts introduced above and discussed in greater detail below may be implemented in any of numerous ways.

Various kinds of sensors can be arranged in and around indoor spaces to detect sensor data regarding the environment in the indoor spaces. For example, temperature sensors, carbon dioxide (CO₂) sensors, and volatile organic compound (VOC) sensors can be positioned in and around indoor spaces to respectively measure and output data regarding temperature, CO₂, and VOCs, respectively. The sensor data can be received directly from the sensors, or via one or more devices or networks connected with the devices.

Various operations can be performed on the sensor data to generate metrics regarding use of the indoor spaces. For example, cognitive indices can be determined from the sensor data to provide objective measures of performance of users or occupants of the indoor spaces for which the sensor data is detected. However, depending on relative values of the sensor data, the cognitive indices derived from the sensor data may have varying levels of significance for indicating actual performance. For example, in some conditions, CO₂ data can be most effective for indicating actual performance, while in other conditions, other parameters (or combinations of parameters) may be more effective. Further, due to the complexity of monitoring and parsing multiple streams of sensor data in real-time or near real-time, it can be difficult to integrate the sensor data or cognitive indices into a unified cognitive index.

Systems and methods as described herein can enable a unified cognitive index to be efficiently and accurately generated from the cognitive indices corresponding to the various forms of sensor data. For example, one or more algorithms, functions, models rules, policies, heuristics, or other operations can be applied to the cognitive indices to prioritize, select, combine, or otherwise manipulate the cognitive indices depending on the values of the cognitive indices or the sensor data used to generate the cognitive indices. By generating a unified cognitive index, computational efficiency can be increased and power usage can be decreased because fewer computational operations are required downstream of the metric generation to generate reports representing the unified cognitive index, perform analytics on the unified cognitive index, or trigger actions based on the unified cognitive index. This can be effective, for example, where the sensor data is received at different rates, such as in an asynchronous manner, which could otherwise complicate various such downstream operations. Consolidating the data into the unified cognitive index can simplify the rendering of display data (e.g., on a graphical user interface (GUI)) relative to continually rendering display data based on multiple cognitive indices, further facilitating real-time displaying the unified cognitive index.

The unified cognitive index can be used to indicate or trigger various actions to improve systems associated with the indoor spaces, such as to indicate building parameter modifications. For example, heating, ventilation, and cooling (HVAC) systems may be operated in a manner that heats, ventilates, or cools spaces without actually improving occupant performance. The unified cognitive index can be provided so that the HVAC systems can be turned on or off, cycled through operational cycles, or have their setpoints determined in a manner that more accurately promotes occupant performance. This can enable power usage to be decreased.

Occupant performance data can be received, concurrently or asynchronously with relative to the sensor data, and used to update and improve the functions, algorithms, or other operations used to generate the unified cognitive index or various other metrics described herein. For example, the occupant performance data can be compared with the unified cognitive index (e.g., subsequent to standardizing the occupant performance data to match the unified cognitive index), and various constants, weights, or other components of the operations can be updated to decrease differences between the occupant performance data and the unified cognitive index.

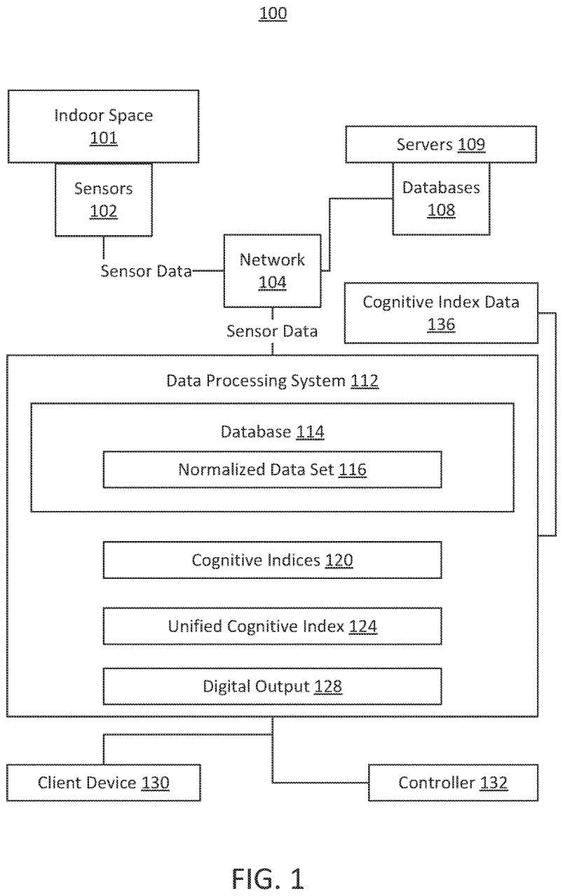

FIG. 1 depicts an example system 100 to perform environmental parameter determination in an indoor environment. The system 100 can include a plurality of sensors 102, which can transmit sensor data via at least one network 104 to any of a variety of databases 108 and data processing systems 112.

The sensors 102 can be arranged in or around at least one indoor space 101. The indoor space 101 can be a space in a building or other structure. The indoor space 101 can be at least partially enclosed, such as by being defined by one or more walls, ceilings, floors, windows, or other structural features. The indoor space 101 can have various components of heating, ventilation, or cooling (HVAC) systems connected with the indoor space, which can be used to flow air into or out of the indoor space 101, heat the air of the indoor space 101, or cool the air of the indoor space 101.

The sensors 102 can include any of a variety of sensors to detect sensor data, such as indoor air composition data, regarding environmental parameters associated with the indoor space 101, including parameters relating to the air in the indoor space 101 and air quality of the air in the indoor space 101. The sensors 102 can detect the sensor data on various schedules. For example, the sensors 102 can detect sensor data periodically; in response to a request from a remote device; during predetermined periods of time (e.g., specific hours or other periods of time during the day, week, or year); in response to trigger conditions (e.g., particular thresholds of the environmental parameters, detecting an occupant entering or exiting the indoor space 101 or being present in the indoor space for a particular period of time); or various other schedules or conditions. Various sensors 102 can detect sensor data on differing schedules or conditions.

The sensors 102 can output the sensor data as one or more sensor data points. Each sensor data point can include a value of the environmental parameter detected by the sensor 102. The sensor data point can include one more identifiers associated with the value. For example, the sensors 102 can assign identifiers to the value, such as a time stamp at which the value was detected, an identifier of the sensor 102 that detected the sensor data, a data type of the sensor data (e.g., the type of parameter, such as temperature, CO₂, or VOC data, or a unit of the parameter, such as Fahrenheit or parts per million (ppm)).

The sensors 102 can output the sensor data synchronously or asynchronously with respect to detecting the sensor data. For example, the sensors 102 can output each sensor data point as it is detected, or output the sensor data points in batches of multiple sensor data points. The sensors 102 can include various wired or wireless communications electronics to facilitate outputting the sensor data.

The sensors 102 can include various sensor for detecting air composition data, such as particulate sensors, light sensors, pressure sensors, motion sensors, or other sensors that can detect data regarding the air in the indoor space. The sensors 102 can include at least one temperature sensor 102, at least one CO₂ sensor 102, and at least one VOC sensor 102.

As such, the sensor data can indicate various metrics regarding the indoor space 101, including metrics regarding indoor air composition data of air in the indoor space 101. For example, the metrics can include a first metric corresponding to temperature, a second metric corresponding to VOCs, and a third metric corresponding to CO₂. Various other metrics may also be indicated by the sensor data for various other parameters regarding the indoor space 101, such as metrics for particulate concentrations, light levels, pressure, motion, or occupant detection.

The sensor data can be transmitted using the at least one network 104. The network 104 may be any type or form of network and may include any of the following: a point-to-point network, a broadcast network, a wide area network, a local area network, a telecommunications network, a data communication network, a computer network, an ATM (Asynchronous Transfer Mode) network, a SONET (Synchronous Optical Network) network, a SDH (Synchronous Digital Hierarchy) network, a wireless network and a wireline network. The network 104 may include a wireless link, such as an infrared channel or satellite band. The topology of the network 104 may include a bus, star, or ring network topology. The network 104 may include mobile telephone networks using any protocol or protocols used to communicate among mobile devices, including advanced mobile phone protocol (“AMPS”), time division multiple access (“TDMA”), code-division multiple access (“CDMA”), global system for mobile communication (“GSM”), general packet radio services (“GPRS”) or universal mobile telecommunications system (“UMTS”). Different types of data may be transmitted via different protocols, or the same types of data may be transmitted via different protocols.

The sensor data can be received by at least one database 108. The database 108 can be implemented by one or more servers 109, which can incorporate features of data processing system 112, computing system 400, or various combinations thereof, as described further herein. The database 108 can be associated with an entity that manages one or more of the sensors 102, the data processing system 112, or various combinations thereof. For example, the database 108 can be associated with a first entity that manages one or more of the sensors 102, such as a manufacturing of the one or more sensors, and the data processing system 112 can be managed by a second entity. Each sensor 102 (or type of sensor 102) can be associated with a respective database 108. The database 108 (e.g., one or more servers 109 that implement the database 108) can perform various data cleaning, normalization, or standardization operations on the sensor data.

The data processing system 112 can include at least one logic device such as a computing device having a processor to communicate via the network 104, for example with the sensors 102 or the database 108. The data processing system 112 can include at least one computation resource, server, processor or memory. For example, the data processing system 112 can include a plurality of computation resources or servers located in at least one data center. The data processing system 112 can include multiple, logically-grouped servers and facilitate distributed computing techniques. The logical group of servers may be referred to as a data center, server farm or a machine farm. The servers can also be geographically dispersed. A data center or machine farm may be administered as a single entity, or the machine farm can include a plurality of machine farms. The servers within each machine farm can be heterogeneous—one or more of the servers or machines can operate according to one or more type of operating system platform.

Servers in the machine farm can be stored in high-density rack systems, along with associated storage systems, and located in an enterprise data center. For example, consolidating the servers in this way may improve system manageability, data security, the physical security of the system, and system performance by locating servers and high performance storage systems on localized high performance networks. Centralization of all or some of the data processing system 112 components, including servers and storage systems, and coupling them with advanced system management tools allows more efficient use of server resources, which saves power and processing requirements and reduces bandwidth usage.

The data processing system 112 can obtain the sensor data (e.g., indoor air composition data) via the network 104. For example, the data processing system 112 can transmit a request for the sensor data (or particular subsets of the sensor data) to the database 108 via the network 104 according to various predetermined schedules, or responsive to receiving a request for the sensor data. As an example, the data processing system 112 can transmit a request for (and in turn receive) the sensor data each minute. The data processing system 112 can obtain the sensor data from the sensors 102 via the network 104 (e.g., without relying on the database 108 as an intermediary connection for receiving the sensor data), including by transmitting requests to respective sensors 102 or having the sensor data pushed from the sensors 102. The data processing system 112 can receive sensor data for each of a variety of metrics simultaneously, not simultaneously (e.g., according to different schedules), periodically, or various combinations thereof at various points in time.

The data processing system 112 can obtain sensor data indicative of one or more particular metrics. For example, the data processing system 112 can obtain, via the network 104, at least one of first indoor air composition data indicative of a first metric (e.g., temperature), second indoor air composition data indicative of a second metric (e.g., VOCs), and third indoor air composition data indicative of a third metric (e.g., CO₂).

The data processing system 112 can store the sensor data in a database 114. The database 114 can incorporate features of the database 108. The database 114 can be one or more of the databases 108. Storing the data can include, for example, performing an extra-transform-load process, in which the data is extracted by being obtained via the network 104, transformed by having data normalization operations performed, and then loaded into the database 114.

The data processing system 112 can store (e.g., load) the sensor data in the database 114 as a normalized data set 116. For example, the data processing system 112 can perform various data normalization (e.g., cleaning, standardization) operations on the sensor data. By performing data normalization on the sensor data in the database 114 to generate the normalized data set 116, the data processing system 112 can more efficiently generate cognitive indices based on the normalized data set 116, which can, for example, allow for real-time or near real-time cognitive index generation.

The data normalization can include various operations to standardize the values of the sensor data or unit labels associated with the sensor data, such as to transform the sensor data into a predetermined format based on the type of sensor data. The type of the sensor data can indicate the environmental parameter or metric that the sensor data indicates. For example, sensor data for temperature can have a temperature type. The data processing system 112 can identify the type of the sensor data (e.g., based on an identifier indicating the source or sensor 102 of the sensor data, or by parsing the unit label provided with the sensor data), and modify the format of the sensor data to have a predetermined format for the normalized data set 116. For example, temperature data may be received having a “TMP” unit label, which the data processing system 112 can modify to “TEMP.” Temperature data can be received in one unit (e.g., Celsius), which the data processing system 112 can modify to another unit (e.g., Fahrenheit). The normalized data set 116 can be arranged with rows corresponding to time stamps and columns corresponding to the sensor data values and the unit labels corresponding to the sensor data values, along with various other data that may be received with or associated with the sensor data, such as identifiers of sensors 102 or the indoor space 101 associated with the sensors 102 or sensor data. For example, a first subset of the normalized data set 116 can correspond to a first indoor space 101, and a second subset of the normalized data set 116 can correspond to a second indoor space 101. Each subset can be identified and retrieved based on an identifier of the subset (which can correspond to an identifier of the respective indoor space 101).

The data normalization that the data processing system 112 performs can include identifying missing values and modifying missing values according to one or more predetermined rules. Missing values can occur, for example, where various sensors 102 provide sensor data in accordance with differing schedules, such that the sensor data from a first sensor 102 may be provided for a particular point in time, but not from a second sensor 102, or where particular sensors 102 may be offline or may output erroneous data at particular points in time. The data processing system 112 can identify a missing value and assign a null value to the value in the normalized data set 116 corresponding to the missing value. By assigning null values to missing values, the data processing system 112 can enable more accurate and rapid determination of cognitive indices.

The data processing system 112 can generate a plurality of cognitive indices 120 based on at least one of the sensor data in the normalized data set 116 and the metrics that the sensor data indicate. The cognitive indices 120 can represent objective measures of performance of occupants in the indoor space 101. The cognitive indices 120 can identify a level of cognitive decline in a human present in the indoor environment. The cognitive indices 120 can be derived, for example, from historical measurements of performance of occupants under analogous conditions as those represented by the sensor data.

The data processing system 112 can generate at least one cognitive index 120 for at least one indoor space 101. The data processing system 112 can generate multiple cognitive indices 120 for at least one indoor space 101. The data processing system 112 can generate multiple cognitive indices 120 for multiple indoor spaces 101, such as to generate aggregate (e.g., average) cognitive indices 120 for a plurality of indoor spaces 101 based on the cognitive indices 120 for each indoor space 101 of the plurality of indoor spaces 101. The data processing system 112 can generate comparisons of cognitive indices 120 between indoor spaces 101, such as to compare a temperature cognitive index 120 for a first indoor space 101 with a temperature cognitive index 120 for a second indoor space 101. The data processing system 112 can generate various measures associated with cognitive indices 120, such as time-averaged or median cognitive indices over a period of time, or rates of change of cognitive indices 120.

The data processing system 112 can generate cognitive indices 120 (or one or more of the cognitive indices 120) responsive to any of a variety of trigger conditions. For example, the data processing system 112 can receive a request from a user (e.g., via a client device, which can include features of or at be at least partially implemented by the data processing system 112, the computing system 400, or various combinations thereof) for one or more cognitive indices 120, and generate the cognitive indices 120 responsive to the request. The request can include, for example, an identifier of one or more indoor spaces 101 or one or more cognitive indices 120 for the one or more indoor spaces 101. The trigger conditions can include a predetermined schedule for generating the cognitive indices 120 being satisfied; for example, the data processing system 112 can generate the cognitive indices 120 periodically, such as every minute, hour, or day. The trigger conditions can include timings associated with usage of the indoor space 101; for example, where the indoor space 101 is of an office building, the data processing system 112 can generate the cognitive indices 120 at the start of a typical workday usage period (e.g., at 8 AM) and end of the workday typical usage period (e.g., 6 PM).

The data processing system 112 can receive sensor data from sensors 102 that monitor occupancy of particular indoor spaces 101, such as motion sensors, proximity sensors, or access controller (e.g., card or badge readers), and generate the cognitive indices 120 responsive to an occupancy trigger condition, such as sensor data indicating an occupant entering (or being present in) the indoor space 101, a threshold number of occupants entering (or being present in) the indoor space 101, an occupant exiting the indoor space 101, or a threshold number of occupants exiting the indoor space 101. For example, the data processing system 112 can request sensor data indicative of occupancy at a relatively infrequent rate (e.g., once per hour), and request sensor data for temperature, CO₂, and VOC metrics for a particular indoor space 101 responsive to the occupancy indicating that a threshold number of occupants are present in the particular indoor space 101 (e.g., at a relatively frequency rate responsive to determining that the threshold number of occupants are present in the particular indoor space 101, such as once per minute).

The data processing system 112 can generate cognitive indices 120 by applying various operations to the normalized data set 116 or one or more subsets of data of the normalized data set 116. The operations can include one or more rules, policies, models, algorithms, regressions, functions, heuristics, or various combinations thereof. The operations can receive the data of the normalized data set 116 as an input and generate an output responsive to the input. The data processing system 112 can apply specific operations to each type of sensor data based on the type of the sensor data, such as to apply a first operation to temperature data to generate a cognitive index 120 based on the temperature data, a second operation to CO₂ data to generate a cognitive index 120 based on the CO₂ data, and a third operation to VOC data to generate a cognitive index 120 based on the VOC data. The data processing system 112 can generate the cognitive indices 120 simultaneously or at different points in time.

The data processing system 112 can generate the cognitive indices 120 to be normalized with respect to the type of sensor data. For example, the data processing system 112 can generate the cognitive indices 120 to be on a same scale for each type of sensor data, such as percentage scale, a 0 to 1 scale, a 0 to 100 scale, or various other normalized scales. For example, the data processing system 112 can identify a value of the cognitive index 120 (before normalization) that corresponds to no effect on performance and modify the operations performed to generate the cognitive index 120 so that a value of 100 is assigned to the identified value of the cognitive index 120, and identify a value of the cognitive index 120 (before normalization) that corresponds to a maximum possible or expected effect on performance and modify operations performed to generate the cognitive index 120 so that a minimum threshold value is assigned to the identified value, such as a threshold value of 0 or 50. This can enable the scale of the cognitive indices 120, independent of the type of the sensor data used to generate the cognitive indices 120, to be consistent, such as to assign a value of 100 to indicate no effect on performance and a value of 50 to indicate a minimum level of effect on performance. As such, the data processing system 112 can generate, from each type of sensor data, cognitive indices 120 that can be compared or combined in various ways (e.g., weighted averages, heuristics, algorithms) across the types of sensor data.

The data processing system 112 can generate the cognitive indices 120 by transmitting a query to the database 114 that causes the database 114 to output the cognitive indices 120, or to output sensor data that the query then applies one or more operations to in order to generate the cognitive indices 120. For example, the data processing system 112 can generate the query to indicate instructions to request particular sensor data from the normalized data set 116 corresponding to the indoor space(s) 101 for which the cognitive indices 120 are to be generated and one or more points in time for which the cognitive indices 120 are to be generated. The data processing system 112 can include instructions that cause the database 114 to perform the operations on the particular sensor data, or the query itself can be a script or other function or code that performs the operations on the particular sensor data responsive to receiving the particular sensor data.

The data processing system 112 can dynamically select the operations (e.g., functions) to perform to generate the cognitive indices 120 based on the values of the sensor data of the normalized data set 116. For example, the data processing system 112 can select a first function to apply sensor data to responsive to the sensor data having a value that is greater than a first threshold, and a second function responsive to the sensor data having a value that is less than a second threshold. This can enable the data processing system 112 to more accurately generate the cognitive indices 120.

For example, to generate the cognitive index 120 based on temperature (e.g., the first metric of the indoor space 101), the data processing system 112 can apply the sensor data for temperature from the normalized data set 116 as input to a function, such as a polynomial function. The polynomial function can be, for example, a first-order polynomial function (e.g., linear function), a second-order polynomial function (e.g., quadratic function), a third-order polynomial function (e.g., cubic function), or various other polynomial function (e.g., functions of the form a₁xⁿ+a₂xⁿ⁻¹+a₃xⁿ⁻²+aₙ₋₁x+aₙ. The constants (a₁ . . . aₙ) can be determined, for example, by the data processing system 112 (or a remote device) applying regressions or other modeling operations on historical data that includes sensor data and cognitive index data, as well as normalization to set the cognitive indices 120 outputted by the polynomial function to a predetermined scale.

For example, the operations performed to determine the cognitive index 120 from temperature can include:

P=0.0033*T³−0.3445*T²+10.377*T+3.4193
if P>100, P=100
   where P is the cognitive index 120 (e.g., productivity relative to maximum value, the maximum value corresponding to P=100), and T is the temperature in Celsius.

To generate the cognitive index 120 based on VOCs (e.g., the second metric of the indoor space 101), the data processing system 112 can apply the VOC sensor data from the normalized data set 116 as input to a polynomial function, including to a linear function. For example, the operations that the data processing system 112 performs to determine the cognitive index 120 from VOC sensor data can include a function based on a 13 percent decrease in cognitive function with 500 μg/m³ increase in VOCs and no decrease in cognitive function (e.g., the value of the cognitive index 120 is 100 percent) at 15 μg/m³.

To generate the cognitive index 120 based on CO₂ (e.g., the third metric of the indoor space 101), the data processing system 112 can apply the CO₂ sensor data from the normalized data set 116 as input to a polynomial function, including to a linear function. For example, the operations that the data processing system 112 performs to determine the cognitive index 120 from CO₂ sensor data can include a function based on a 12 percent decrease in cognitive function with 400 ppm increase in CO₂, with 500 ppm at 100 percent, such as the following operations (e.g., a piecewise decision tree function dependent on the value of CO₂ using the following operations):
if (C*−0.0352+115.8)>100 then P=100
else if (C>2500) then P=(2500*−0.0352+115.8)
else P=C*−0.0352+115.8
   where C is the value of CO₂ (e.g., in ppm) and P is the cognitive index 120 (e.g., productivity relative to maximum value, the maximum value corresponding to P=100).

The data processing system 112 can generate, based on at least two of the cognitive indices 120, a unified cognitive index 124. The unified cognitive index 124 can provide a single, accurate value representative of how the conditions in the indoor space 101 can affect performance of occupants of the indoor space 101. By consolidating the cognitive indices 120 to the unified cognitive index 124, the data processing system 112 can have improved operation by reducing the number of downstream operations needed to be performed on the unified cognitive index 124 (e.g., by a factor of three relative to using each of three cognitive indices from temperature, VOC, and CO₂ data), including for generating and rendering outputs presenting the unified cognitive index 124, or for triggering actions responsive to the unified cognitive index 124.

The data processing system 112 can perform at least one of comparing the cognitive indices 120 with one another (e.g., compare at least two cognitive indices 120) and comparing the cognitive indices with a threshold to select the cognitive indices 120 as candidate values for the unified index 124. For example, the data processing system 112 can compare each of the cognitive indices 120 with a minimum threshold, and discard (e.g., not consider as a candidate value; not determine the unified cognitive index 124 based on) one or more cognitive indices 120 that are less than (or less than or equal to) the minimum threshold (e.g., cognitive indices 120 that do not satisfy the threshold). The minimum threshold can be associated with a value below which cognitive indices have not been measured, or below which cognitive indices are no longer realistic. For example, the minimum threshold can be value between 0 and 70. The minimum threshold can be between 40 and 60. The minimum threshold can be 50. The minimum threshold can depend on the type of sensor data.

For example, the data processing system 112 can apply averages, weighted averages, decision tree selection functions, Bayesian selection functions, threshold-based selection functions, or various other operations to the cognitive indices 120 generate the unified cognitive index 124.

Responsive to one or more cognitive indices 120 being greater than the threshold, the data processing system 112 can compare the cognitive indices 120 that are greater than the threshold with one another, and select, as the unified cognitive index 124, the lesser of the cognitive indices 120 that are greater than the threshold. For example, the data processing system 112 can generate the unified cognitive index 124 by selecting one of the cognitive indices 120 that is (1) greater than the threshold and (2) less than the other(s) of the cognitive indices 120. As such, the cognitive index 120 corresponding to the parameter (temperature, VOCs, CO₂) having the greatest impact on performance (e.g., greatest reduction in performance) can be selected as the unified cognitive index 124. This can correspond to the data processing system 112 performing a weighting of the cognitive indices 120, in which a weight of 1 is assigned to the cognitive index 120 having the lesser value and a weight of 0 is assigned to the cognitive index (or indices) 120 having the greater values. For example, various weightings of the cognitive indices 120 can be performed (e.g., by the data processing system 112 determining weights to apply to each cognitive index 120 using regressions or other models of historical data) to generate the unified cognitive index 124, such as predetermined weights or weights that depend on the values of the cognitive indices 120.

For example, the data processing system 112 can determine that a first cognitive index 120 based on temperature has a value of 60, a second cognitive index 120 based on VOCs has a value of 65, and a third cognitive index 120 based on CO₂ has a value of 45. The data processing system 112 can compare the cognitive indices 120 to a minimum threshold of 50, and discard the third cognitive index 120 (or select the first and second cognitive indices 120) responsive to the third cognitive index 120 being less than the minimum threshold (or the first and second cognitive indices 120 being greater than the minimum threshold). The data processing system 112 can compare the first and second cognitive indices 120 with one another to determine that the first cognitive index 120 is less than the second cognitive index 120, and generate the unified cognitive index 124 to have the value of the first cognitive index 120.

The data processing system 112 can assign priorities to the cognitive indices 120 to generate the unified cognitive index 124. For example, the CO₂-based cognitive index 120 can have a greater priority than the temperature-based cognitive index 120, which the data processing system 112 can use to assign a higher weight to the CO₂-based cognitive index 120 than the temperature-based cognitive index 120. The data processing system 112 can assign priorities to the cognitive indices 120 based on the values of the cognitive indices 120 (or the underlying sensor data); for example, CO₂ may have a stronger impact on performance at relatively low cognitive index 120 values than temperature, but not at relatively higher values. As such, if the cognitive indices 120 are each in a first range (e.g., from the minimum threshold to an intermediate threshold), the data processing system 112 can generate the unified cognitive index 124 to be the cognitive index 120 having the higher priority in the first range.

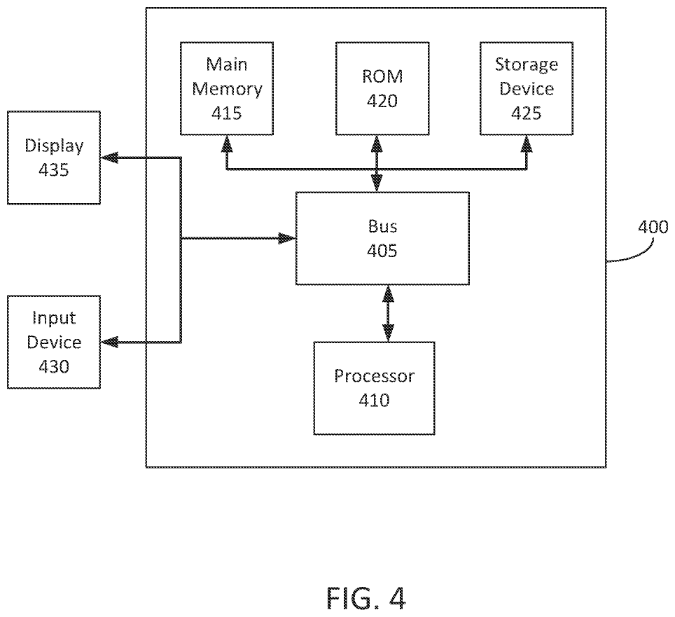

The data processing system 112 can generate, responsive to the unified cognitive index 124, a digital output 128 that corresponds to the unified cognitive index 124. The digital output 128 can include a percentage value representing the unified cognitive index 124. The data processing system 112 can provide the digital output 128 for rendering by a display of a client device 130 (e.g., computing device 400 described with reference to FIG. 4). The data processing system 112 can generate and provide the digital output 128 responsive to a request for the digital output 128, such as a request received from the client device 130 (e.g., from a user interface presented by the client device 130). The client device 130 can use the digital output to render and present one or more display images that include the digital output 128.

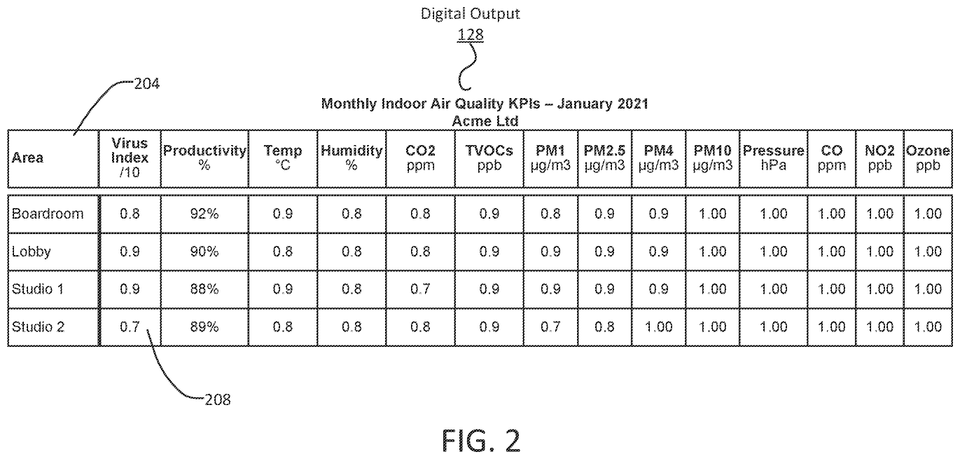

For example, as depicted in FIG. 2, the data processing system 112 can generate the digital output 128 to include at least one column 204 and at least one row 208. Each row 208 can be associated with a respective indoor space 101. Each column 204 can be associated with the unified cognitive index 124 for the corresponding indoor space 101 or sensor data (e.g., air composition data) for the corresponding indoor space 101. For example, the columns 204 can include sensor data such as virus, temperature, humidity, CO₂ VOCs, PM2.5, pressure, CO (carbon monoxide), NO₂ (nitrogen dioxide), and ozone values. The data processing system 112 can compare the values of the unified cognitive index 124 or the sensor data to respective thresholds, and adjust how the data is displayed (e.g., by controlling colors, text sizes, or text formatting) responsive to the comparisons. For example, if the unified cognitive index 124 for a particular indoor space 101 is greater than a first display threshold (e.g., greater than 80), the data processing system 112 can cause the unified cognitive index 124 to be displayed using a green color.

The data processing system 112 can provide the unified cognitive index 124 to devices to one or more controllers 132. The controllers 132 can be, for example, devices that control operation of HVAC devices or systems, such as controllers of heaters, air conditioners, fans, pumps, or valves, including but not limited to thermostats. The controllers 132 can be controllers of lighting systems. The controllers 132 can adjust operation of controlled devices responsive to the unified cognitive index 124. For example, the controller 132 for an HVAC device can store at least one setpoint responsive to which the HVAC device causes heating (or cooling), such as to activate or deactivate a fan, pump, or valve. The setpoints can be temperature-based setpoints. The controller 132 can adjust the setpoints responsive to the unified cognitive index 124, such as to increase a setpoint for activating a cooling process responsive to the unified cognitive index 124 being greater than a performance threshold (e.g., a threshold indicative of sufficient performance levels). For example, the controller 132 can have a setpoint of 74 degrees Fahrenheit, above which the controller 132 causes a cooling process to occur. Responsive to determining that the unified cognitive index 124 satisfies the performance threshold while the temperature is 74 degrees Fahrenheit (even if the unified cognitive index 124 did not take into account temperature data), the controller 132 can increase the setpoint (e.g., to 75 degrees Fahrenheit), which can avoid power usage for cooling between 74 and 75 degrees Fahrenheit without allowing performance to fall below the performance threshold.

The data processing system 112 can adjust how the cognitive indices 120 are generated using cognitive index data 136. For example, the data processing system 112 can receive cognitive index data 136 corresponding to measurements of occupants of indoor spaces 101, together with sensor data measured for the indoor spaces 101. The cognitive index data 136 can be received from any of a variety of sources, databases, or entities, including but not limited to the data processing system 112 can apply various regressions, machine learning models, or other operations to the cognitive index data 136 and the operations used to generate the cognitive indices 120 to update the operations used to generate the cognitive indices 120. For example, the data processing system 112 can apply the sensor data measured for the indoor spaces 101 as an input to the operations (e.g., polynomial functions) described above to generate candidate outputs, compare the candidate outputs with the cognitive index data 136, and modify the constants of the polynomial functions responsive to the comparison. As such, the data processing system 112 can continually improve the accuracy of the cognitive index determination.

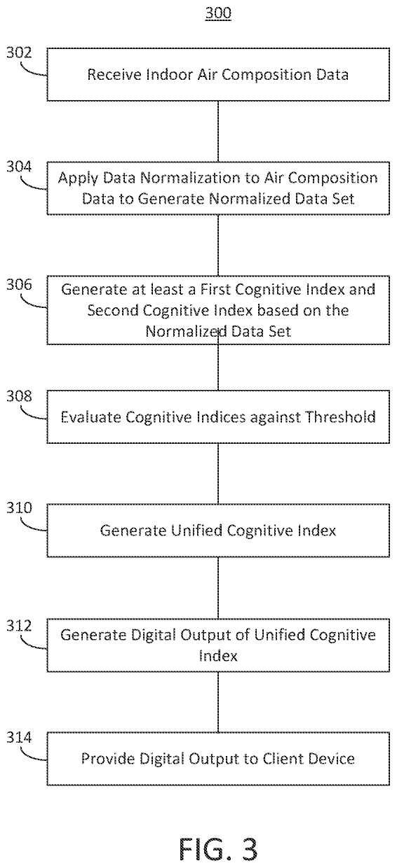

FIG. 3 depicts an example of a method 300 of environmental parameter determination. The method 300 can be performed using various systems and devices described herein, including but not limited to the system 100 and the computer system 400. The method 300 or portions or steps thereof can be performed responsive to various conditions, such as requests for cognitive indices, such as for generating and presenting digital outputs of cognitive indices, or for controlling operation of devices that rely on the cognitive indices.

At 302, indoor air composition data is received. The indoor air composition data can include data from a plurality of sensors, such as temperature, VOC, and CO₂ sensors. The indoor air composition data can be obtained via a network. The indoor air composition data can be received from the sensors via the network, from one or more databases that receive the indoor air composition data from the sensors, or various compositions thereof. The indoor air composition data can be obtained responsive to a request for indoor air composition data, such as a request for indoor air composition data for a particular indoor space or a particular plurality of indoor spaces, or responsive to the sensors or the database outputting the indoor air composition data. For example, indoor air composition data can be requested for temperature data, VOC data, and CO₂ data for a particular indoor space.

At 304, data normalized is applied to the indoor air composition data to generate a normalized data set. The data normalization can be applied to standardize the indoor air composition data, such as to assign predetermined unit labels to the indoor air composition data (e.g., assign “TEMP” instead of “TMP” as a unit label for temperature data). The data normalization can include modifying units (e.g., Fahrenheit to Celsius) or a scale of the indoor air composition data (e.g., filtering out data values that are above maximum thresholds or below minimum thresholds). The data normalization can include assigning null values to missing values.

At 306, at least a first cognitive index and a second cognitive index are generated based on the normalized data set. The cognitive indices can be generated by applying one or more functions, algorithms, rules, models, or other operations to the data of the normalized data set. For example, the normalized temperature data can be applied as input to a temperature-specific function to generate the first cognitive index, the normalized VOC data can be applied as input to a VOC-specific function to generate the second cognitive index, and the normalized CO₂ data can be applied as input to a CO₂-specific function to generate a third cognitive index. For example, specific linear or polynomial functions can be applied to each respective normalized data to generate the respective cognitive indices. The cognitive indices can be generated simultaneously or according to differing periods or schedules. The cognitive indices can be generated to be on a normalized scale (e.g., a percentage scale from 0 to 100).

At 308, the cognitive indices are evaluated against a threshold. The threshold can be a minimum threshold indicative of a point below which occupant performance cannot be reasonably measured or further decreased. For example, the minimum threshold can be 50 on the 0 to 100 scale, such that if any of the first, second, or third cognitive indices have values less than 50, they may be discarded or otherwise not considered for generating the unified cognitive index (or the cognitive indices that satisfy the threshold can be considered as candidate values for generating the unified cognitive index).

At 310, a unified cognitive index is generated. The unified cognitive index can be generated based on the cognitive indices that satisfied the threshold. For example, each cognitive index that satisfied the threshold can be compared with the other cognitive index (or indices) that satisfied the threshold to identify the cognitive index having the lowest value (yet still satisfying the threshold), and the cognitive index having the lowest value can be selected as the unified cognitive index. The unified cognitive index can be generated by applying a weighted average function to the cognitive indices that satisfied the threshold, such as a weighted average that weights each cognitive index based on at least one of a type of the cognitive index, a priority assigned to the cognitive index, or the value of one or more of the cognitive indices.

At 312, a digital output of the unified cognitive index is generated. The digital output can include the value of the unified cognitive index. The digital output can include a table, chart, or other graphic representing the unified cognitive index. The digital output can include indoor air composition data or other sensor data associated with the indoor space(s) for which the unified cognitive index was generated. For example, the digital output can include a table having rows corresponding to respective indoor spaces and columns corresponding to values of unified cognitive indices and sensor data.

At 314, the digital output is provided to a client device. For example, the digital output can be provided to a client device that requested the digital output. The digital output can be provided via a network connection between one or more devices that generated the unified cognitive index and the client device.

FIG. 4 is a block diagram of an example computer system 400. The computer system or computing device 400 can include or be used to implement the system 100, or its components such as the data processing system 102. The computing system 400 includes a bus 405 or other communication component for communicating information and a processor 410 or processing circuit coupled to the bus 405 for processing information. The computing system 400 can also include one or more processors 410 or processing circuits coupled to the bus for processing information. The computing system 400 also includes main memory 415, such as a random access memory (RAM) or other dynamic storage device, coupled to the bus 405 for storing information, and instructions to be executed by the processor 410. The main memory 415 can be or include the database 114. The main memory 415 can also be used for storing position information, temporary variables, or other intermediate information during execution of instructions by the processor 410. The computing system 400 may further include a read only memory (ROM) 420 or other static storage device coupled to the bus 405 for storing static information and instructions for the processor 410. A storage device 425, such as a solid state device, magnetic disk or optical disk, can be coupled to the bus 405 to persistently store information and instructions. The storage device 425 can include or be part of the database 114.

The computing system 400 may be coupled via the bus 405 to a display 435, such as a liquid crystal display, or active matrix display, for displaying information to a user. An input device 430, such as a keyboard including alphanumeric and other keys, may be coupled to the bus 405 for communicating information and command selections to the processor 410. The input device 430 can include a touch screen display 435. The input device 430 can also include a cursor control, such as a mouse, a trackball, or cursor direction keys, for communicating direction information and command selections to the processor 410 and for controlling cursor movement on the display 435. The display 435 can be part of the data processing system 102, the client device 130 or other component of FIG. 1, for example.

The processes, systems and methods described herein can be implemented by the computing system 400 in response to the processor 410 executing an arrangement of instructions contained in main memory 415. Such instructions can be read into main memory 415 from another computer-readable medium, such as the storage device 425. Execution of the arrangement of instructions contained in main memory 415 causes the computing system 400 to perform the illustrative processes described herein. One or more processors in a multi-processing arrangement may also be employed to execute the instructions contained in main memory 415. Hard-wired circuitry can be used in place of or in combination with software instructions together with the systems and methods described herein. Systems and methods described herein are not limited to any specific combination of hardware circuitry and software.

Although an example computing system has been described in FIG. 4, the subject matter including the operations described in this specification can be implemented in other types of digital electronic circuitry, or in computer software, firmware, or hardware, including the structures disclosed in this specification and their structural equivalents, or in combinations of one or more of them.

The subject matter and the operations described in this specification can be implemented in digital electronic circuitry, or in computer software, firmware, or hardware, including the structures disclosed in this specification and their structural equivalents, or in combinations of one or more of them. The subject matter described in this specification can be implemented as one or more computer programs, e.g., one or more circuits of computer program instructions, encoded on one or more computer storage media for execution by, or to control the operation of, data processing apparatuses. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. While a computer storage medium is not a propagated signal, a computer storage medium can be a source or destination of computer program instructions encoded in an artificially generated propagated signal. The computer storage medium can also be, or be included in, one or more separate components or media (e.g., multiple CDs, disks, or other storage devices). The operations described in this specification can be implemented as operations performed by a data processing apparatus on data stored on one or more computer-readable storage devices or received from other sources.

The terms “data processing system” “computing device” “component” or “data processing apparatus” encompass various apparatuses, devices, and machines for processing data, including by way of example a programmable processor, a computer, a system on a chip, or multiple ones, or combinations of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, cloud computing, distributed computing and grid computing infrastructures. For example, the database 114 and other data processing system 112 components can include or share one or more data processing apparatuses, systems, computing devices, or processors.

A computer program (also known as a program, software, software application, app, script, or code) can be written in any form of programming language, including compiled or interpreted languages, declarative or procedural languages, and can be deployed in any form, including as a stand-alone program or as a module, component, subroutine, object, or other unit suitable for use in a computing environment. A computer program can correspond to a file in a file system. A computer program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs (e.g., components of the data processing system 112) to perform actions by operating on input data and generating output. The processes and logic flows can also be performed by, and apparatuses can also be implemented as, special purpose logic circuitry, e.g., an FPGA (field programmable gate array) or an ASIC (application specific integrated circuit). Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

The subject matter described herein can be implemented in a computing system that includes a back end component, e.g., as a data server, or that includes a middleware component, e.g., an application server, or that includes a front end component, e.g., a client computer having a graphical user interface or a web browser through which a user can interact with an implementation of the subject matter described in this specification, or a combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system such as system 100 or system 400 can include clients and servers. A client and server are generally remote from each other and typically interact through a communication network (e.g., the network 104). The relationship of client and server arises by virtue of computer programs running on the respective computers and having a client-server relationship to each other. In some implementations, a server transmits data (e.g., data packets representing a digital component) to a client device (e.g., for purposes of displaying data to and receiving user input from a user interacting with the client device). Data generated at the client device (e.g., a result of the user interaction) can be received from the client device at the server (e.g., received by the data processing system 112).

While operations are depicted in the drawings in a particular order, such operations are not required to be performed in the particular order shown or in sequential order, and all illustrated operations are not required to be performed. Actions described herein can be performed in a different order.

The separation of various system components does not require separation in all implementations, and the described program components can be included in a single hardware or software product. For example, the data processing system 112 can be a single component, app, or program, or a logic device having one or more processing circuits, or part of one or more servers of the data processing system 112.

Having now described some illustrative implementations, it is apparent that the foregoing is illustrative and not limiting, having been provided by way of example. In particular, although many of the examples presented herein involve specific combinations of method acts or system elements, those acts and those elements may be combined in other ways to accomplish the same objectives. Acts, elements and features discussed in connection with one implementation are not intended to be excluded from a similar role in other implementations or implementations.

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular may also embrace implementations including a plurality of these elements, and any references in plural to any implementation or element or act herein may also embrace implementations including only a single element. References in the singular or plural form are not intended to limit the presently disclosed systems or methods, their components, acts, or elements to single or plural configurations. References to any act or element being based on any information, act or element may include implementations where the act or element is based at least in part on any information, act, or element.

Any implementation disclosed herein may be combined with any other implementation or embodiment, and references to “an implementation,” “some implementations,” “one implementation” or the like are not necessarily mutually exclusive and are intended to indicate that a particular feature, structure, or characteristic described in connection with the implementation may be included in at least one implementation or embodiment. Such terms as used herein are not necessarily all referring to the same implementation. Any implementation may be combined with any other implementation, inclusively or exclusively, in any manner consistent with the aspects and implementations disclosed herein.

References to “or” may be construed as inclusive so that any terms described using “or” may indicate any of a single, more than one, and all of the described terms. References to at least one of a conjunctive list of terms may be construed as an inclusive OR to indicate any of a single, more than one, and all of the described terms. For example, a reference to “at least one of ‘A’ and ‘B’” can include only ‘A’, only ‘B’, as well as both ‘A’ and ‘B’. Such references used in conjunction with “comprising” or other open terminology can include additional items.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

The systems and methods described herein may be embodied in other specific forms without departing from the characteristics thereof. For example, while the cognitive indices are described primarily in terms of temperature, VOC, and CO₂ data, various other metrics, such as particulates, CO, pressure, and occupancy can be used. The various indices can be data structures that can be organized, managed, and stored by the data processing systems and processors described herein. The foregoing implementations are illustrative rather than limiting of the described systems and methods. Scope of the systems and methods described herein is thus indicated by the appended claims, rather than the foregoing description, and changes that come within the meaning and range of equivalency of the claims are embraced therein.

What is claimed is:

1. A system of environmental parameter determination in an indoor environment, comprising:
a data processing system comprising memory and at least one processor to:
obtain, via a network and from a first sensor, first indoor air composition data that indicates a first metric of an indoor space;
obtain, via the network and from a second sensor, second indoor air composition data that indicates a second metric of the indoor space;
obtain, via the network and from a third sensor, third indoor air composition data that indicates a third metric of the indoor space;
apply a data normalization operation to at least one of the first indoor air composition data, the second indoor air composition data, and the third indoor air composition data to generate a normalized data set for storage in a database, the normalized data set including the first indoor air composition data, the second indoor air composition data, and the third indoor air composition data;
obtain, from the database, the normalized data set to generate at least a first cognitive index and a second cognitive index, each of the first cognitive index and the second cognitive index corresponding to one of the first metric of the indoor space, the second metric of the indoor space, or the third metric of the indoor space;
compare each of the first cognitive index and the second cognitive index with a threshold;
generate, based on the first cognitive index and the second cognitive index, a unified cognitive index for the indoor space;
generate, responsive to the unified cognitive index, a digital output that corresponds to the unified cognitive index; and
provide, from the data processing system, the digital output to a client computing device for display by the client computing device.

2. The system of claim 1, comprising:
the data processing system is to generate the first cognitive index by applying the first metric of the indoor space as input to a polynomial function, the first metric of the indoor space corresponding to temperature data.

3. The system of claim 1, comprising:
the data processing system is to generate the second cognitive index by applying the second metric of the indoor space as input to a linear function, the second metric of the indoor space corresponding to volatile organic compound (VOC) data.

4. The system of claim 1, comprising:
the data processing system is to generate the third cognitive index by applying the third metric of the indoor space as input to a piecewise function, the third metric of the indoor space corresponding to CO₂ data.

5. The system of claim 1, comprising:
the data processing system is to generate the unified cognitive index by selecting one of the first cognitive index or the second cognitive index that is (1) greater than the threshold and (2) less than the other of the first cognitive index or the second cognitive index.

6. The system of claim 1, comprising:
the data processing system is to obtain, from the database, the normalized data set to generate a third cognitive index, the first cognitive index corresponding to the first metric of the indoor space, the second cognitive index corresponding to the second metric of the indoor space, and the third cognitive index corresponding to the third metric of the indoor space.

7. The system of claim 1, comprising:
the data processing system is to apply a data normalization operation to each of the first indoor air composition data, the second indoor air composition data, and the third indoor air composition data to generate the normalized data set.

8. The system of claim 1, comprising:
the data processing system is to:
determine, responsive to comparing the first cognitive index with the threshold, that the first cognitive index does not satisfy the threshold; and
generate, responsive to the first cognitive index not satisfying the threshold, the unified cognitive index based on the second cognitive index and not based on the first cognitive index.

9. The system of claim 1, comprising:
the data processing system is to:
determine, responsive to comparing the first cognitive index with the threshold and the second cognitive index with the threshold, that the first cognitive index satisfies the threshold and the second cognitive index satisfies the threshold;
determine, responsive to determining that the first cognitive index satisfies the threshold and the second cognitive index satisfies the threshold, that the first cognitive index is less than the second cognitive index; and
generate, responsive to determining that the first cognitive index is less than the second cognitive index, the unified cognitive index based on the second cognitive index and not based on the first cognitive index.

10. The system of claim 1, comprising:
the first indoor air composition data and the first metric of the indoor space correspond to temperature data.

11. The system of claim 1, comprising:
the second indoor air composition data and the second metric of the indoor space correspond to CO₂ data.

12. The system of claim 1, comprising:
the third indoor air composition data and the third metric of the indoor space correspond to VOC data.

13. The system of claim 1, comprising:
the first cognitive index corresponds to the first metric of the indoor space, and the second cognitive index corresponds to the second metric of the indoor space.

14. The system of claim 1, comprising:
each of the first cognitive index and the second cognitive index identifies a level of cognitive decline in a human present in the indoor environment.

15. The system of claim 1, comprising:
the digital output corresponding to the unified digital index is a percentage value.

16. The system of claim 1, comprising:
the data processing system is to apply the data normalization operation to generate the normalized data set by modifying the at least one of the first indoor air composition data, the second indoor air composition data, and the third indoor air composition data to correspond to a predetermined numerical scale.

17. The system of claim 1, comprising:
the data processing system is to obtain the first indoor air composition data, the second indoor air composition data, and the third indoor air composition data at least one of simultaneously and periodically.

18. The system of claim 1, comprising:
the data processing system is to generate the first cognitive index and the second cognitive index by providing a query to the database, the query comprising at least a first operation that uses the first metric of the indoor space as input to generate the first cognitive index and a second operation that uses the second metric as input to generate the second cognitive index.

19. A method of environmental parameter determination in an indoor environment, comprising:
receiving, by a data processing system comprising memory and at least one processor, from a first sensor, first indoor air composition data that indicates a first metric of an indoor space;
receiving, by the data processing system, from second first sensor, second indoor air composition data that indicates a second metric of an indoor space;
receiving, by the data processing system, from a third sensor, third indoor air composition data that indicates a first metric of an indoor space;
applying a data normalization operation to at least one of the first indoor air composition data, the second indoor air composition data, and the third indoor air composition data to generate a normalized data set for storage in a database, the normalized data set including the first indoor air composition data, the second indoor air composition data, and the third indoor air composition data;
generating, based on the normalized data set retrieved, from the database, at least a first cognitive index and a second cognitive index, each of the first cognitive index and the second cognitive index corresponding to one of the first metric of the indoor space, the second metric of the indoor space, or the third metric of the indoor space;
evaluating each of the first cognitive index and the second cognitive index against a threshold;
generating, based on the first cognitive index and the second cognitive index, a unified cognitive index for the indoor space;
generating, responsive to the unified cognitive index, a digital output corresponding to the unified cognitive index; and
providing, from the data processing system, the digital output to a client computing device for display by the client computing device.

20. The method of claim 19, comprising:
the first metric of the indoor space is a temperature metric, the second metric of the indoor space is a volatile organic compound (VOC) metric, the third metric of the indoor space is a CO₂ metric, and generating the unified cognitive index comprises applying a plurality of polynomial functions to the first indoor air composition data, the second indoor air composition data, and the third indoor air composition data.
